(19) United States
(12) Patent Application Publication
(10) Pub. No.: US 2024/0206515 A1
(43) Pub. Date: Jun. 27, 2024

(54) Expression of Heme Biosynthesis and Heme Proteins in Edible Filamentous Fungi

(71) Applicant: The Regents of the University of California, Oakland, CA (US)

(72) Inventors: Jay D. Keasling, Berkeley, CA (US); Vayu Maini Rekdal, Berkeley, CA (US)

(73) Assignee: The Regents of the University of California, Oakland, CA (US)

(21) Appl. No.: 18/433,372

(22) Filed: Feb. 5, 2024

Related U.S. Application Data
(63) Continuation of application No. PCT/US22/42816, filed on Sep. 7, 2022.
(60) Provisional application No. 63/241,530, filed on Sep. 7, 2021.

(51) Int. Cl. A23L 31/00; A23J 3/20; A23J 3/22; C12N 1/14; C12R 1/69

(52) U.S. Cl. CPC A23L 31/00; A23J 3/20; A23J 3/227; C12N 1/145; C12R 2001/69

(57) ABSTRACT

Food compositions that mimic flavors and texture of animal meats are made from an edible filamentous fungus genetically engineered to overexpress heme biosynthesis genes to elevate heme levels.

### CROSS-REFERENCES TO RELATED APPLICATIONS

This application is a continuation of PCT/US22/42816; filed: Sep. 7, 2022, which claims priority to U.S. Provisional Application No. 63/241,530; filed: Sep. 7, 2021, the disclosures of which are hereby incorporated by reference in its entirety for all purposes.

### INTRODUCTION

Heme is an essential cofactor in many metabolic processes across all kingdoms of life and is also a key contributor to both the flavor and texture of animal meat. Elevating heme levels has recently emerged as a promising strategy for imparting meat-like flavor and appearance to plant-based meat replacements. For example, the heterologously expressed Soy Leghemoglobin is a key ingredient a plant-based meat formulation which has had widespread commercial success. Despite the promising meat-like flavor and appearance of plant-based products containing elevated heme levels, a general limitation of current plant-based meat replacements is that it is difficult to achieve whole-cut textures that are similar to animal muscles. A majority of plant-based meat replacements are therefore only available in processed or ground formulations and require extensive processing and additives, which decreases the nutritive value of the product and contributes to consumer hesitance.

In contrast to plant protein isolates, organisms such as filamentous fungi have meat-like textures due to their filamentous morphology. The filaments have the ability to mimic muscle fibers in animal tissue, making fungal biomass promising targets to create whole-cut, muscle-like meat replacements with minimal processing. Edible filamentous fungi are delicious, nutritious, sustainable, and have a long history of safe consumption by humans. While fungal biomass has promising textures, it sometimes lacks meat-like flavor and appearance

### SUMMARY OF THE INVENTION

The invention provides food compositions that mimic flavors and texture of animal meats are made from an edible filamentous fungus genetically engineered to overexpress heme biosynthesis genes and/or genes encoding heme proteins to elevate heme levels.

In an aspect the invention provides a composition comprising an edible filamentous fungus genetically engineered to overexpress heme biosynthesis genes (and/or genes encoding heme proteins) to elevate heme levels above a corresponding non-engineered level.

In embodiments:

the genes include aminolevulinic acid synthase (ALAS), aminolevulinic acid dehydratase (ALAD), uroporphyrinogen decarboxylase (UROD), coproporphyrinogen oxidase (CPO), protoporphyrinogenidase (PPO), porphobilinogen deaminase (PBGD), uroporphyrinogen III synthase (UROS) and ferrochelatase (FC) (see FIG. 1), or a subcombination;

the genes include a Heme Regulatory Motif (HRM) mutant in ALAS;

the fungus is selected from Aspergillus oryzae, Neurospora intermedia, and Neurospora crassa;

the fungus is Aspergillus oryzae NSAR1

the fungus is Aspergillus oryzae RIB40

the fungus provides increased heme levels of at least 5, 10 or 15-fold above the non-engineered background strain, as assessed by LC-MS;

the fungus is engineered to express a hemoglobin protein, which acts as a sink for elevated heme inside the fungus;

the fungus is engineered to express a hemoglobin protein, such as Leghemoglobins from soy, alfafa, clove; full-length and hemoglobin domains of flavohemoglobins from Neurospora crassa, Aspergillus oryzae, Saccharomyces cerevisiae; or hemoglobins from cow, pig, chicken, salmon, and tuna;

the composition is engineered with flavor components sufficient to mimic flavors, apperances, and textures of a meat selected from beef, chicken or pork;

the composition is formatted as a mimic and/or substitute of a meat product selected from ground beef, beef steak, bacon and sausage; and/or

the fungus is growable on an inexpensive substrate, such as cellulosic biomass, molasses or food waste.

In an aspect, the invention provides a method of making an edible filamentous fungus, the method comprising genetically engineering the fungus to overexpress heme biosynthesis genes and/or one or more heme proteins to elevate heme levels above a corresponding non-engineered level.

The invention encompasses all combinations of the particular embodiments recited herein, as if each combination had been laboriously recited.

### BRIEF DESCRIPTION OF THE DRAWINGS

FIG. 1. Heme biosynthetic pathway in filamentous fungi; see: Franken, A. C. W., Lokman, B. C., Ram, A. F. J. et al. Heme biosynthesis and its regulation: towards understanding and improvement of heme biosynthesis in filamentous fungi. Appl Microbiol Biotechnol 91, 447-460 (2011).

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

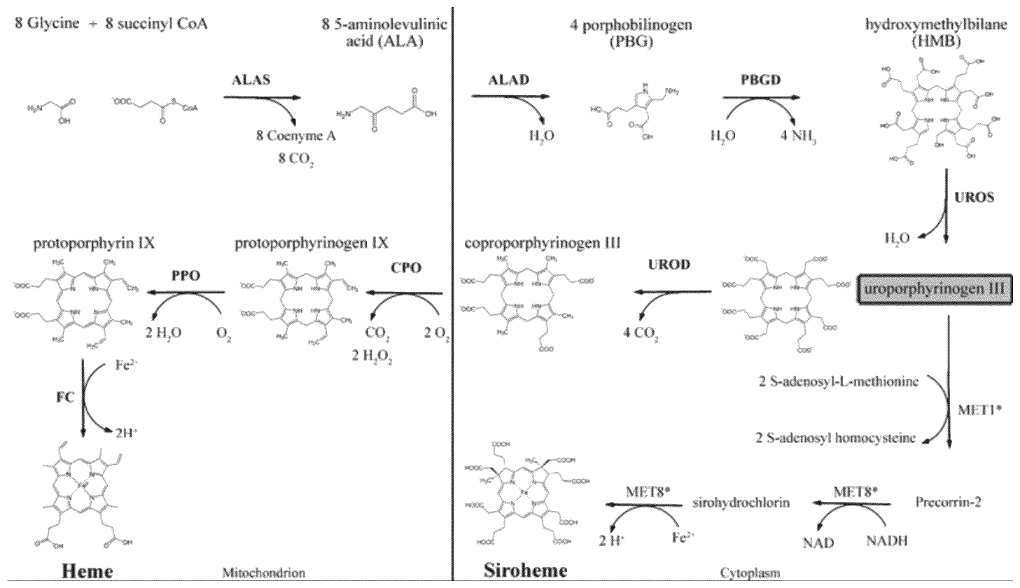

The invention provides genetic engineering of edible filamentous fungi, providing improved flavor and appearance of these organisms for applications in food. In proof-of-principle examples, we have overexpressed heme biosynthesis genes in edible filamentous fungi to elevate heme levels beyond endogenous levels. Overexpression of key biosynthetic enzymes, including a Heme Regulatory Motif (HRM) mutant in ALAS, as well as ALAD, UROD, CPO, PPO, PBGD, UROS, and FC, in several different subcombinations (see FIG. 1 for biosynthetic pathway) in Aspergillus oryzae, Neurospora intermedia, and Neurospora crassa strains, including the edible filamentous fungus Aspergillus oryzae NSAR1 and Aspergillus oryzae RIB40, significantly increased heme levels in excess of 5, 10 and 15-fold above the non-engineered background strain, as assessed by LC-MS. The fungal biomass is red in appearance and is used in meat replacements, including burgers, sausages, filets, bacon, and other whole-cut formulations.

Our expression protocols are based on well-established toolkit methodologies, e.g. David Lubertozzi, Jay D. Keasling, Biotechnology Advances, 27 (1), 2009, p.53-75, Developing Aspergillus as a host for heterologous expression; Khomaizon et al., Methods Enzymol. 2012;517:241-60: A toolkit for heterologous expression of metabolic pathways in Aspergillus oryzae; and Mózsik et al, ACS Synth. Biol. 2021, 10, 11, 2850-2861; Modular Synthetic Biology Toolkit for Filamentous Fungi.

Additional embodiments of the engineered organisms include combinations of the heme biosynthetic enzymes, and/or the addition of a hemoglobin protein, which acts as a sink for the elevated heme inside the fungal cells. Suitable hemoglobin proteins include Leghemoglobins from soy, alfafa, clove; full-length and hemoglobin domains of flavohemoglobins from Neurospora crassa, Aspergillus oryzae, Saccharomyces cerevisiae; hemoglobins from cow, pig, chicken, salmon, and tuna.

The invention provides production of engineered fungal biomass at a large scale using cheap nutrient sources and to transform the biomass into meat replacements that have meat-like appearance, texture, and flavor, with minimal processing. The invention can build all the necessary meat-like flavors into an edible filamentous fungus, meaning nothing has to be added exogenously to meat-like products. The extensive use of additives to accomplish meat-like flavor is a current limitation of meat replacement products.

1. A composition comprising an edible filamentous fungus genetically engineered to overexpress heme biosynthesis genes and/or genes encoding heme proteins to elevate heme levels above a corresponding non-engineered level.

2. The composition of claim 1, wherein the genes include aminolevulinic acid synthase (ALAS), aminolevulinic acid dehydratase (ALAD), uroporphyrinogen decarboxylase (UROD), coproporphyrinogen oxidase (CPO), protoporphyrinogenidase (PPO), porphobilinogen deaminase (PBGD), uroporphyrinogen III synthase (UROS) and ferrochelatase (FC), or a subcombination thereof.

3. The composition of claim 1, wherein the genes include aminolevulinic acid synthase (ALAS) and aminolevulinic acid dehydratase (ALAD).

4. The composition of claim 1, wherein the genes include aminolevulinic acid synthase (ALAS), aminolevulinic acid dehydratase (ALAD), uroporphyrinogen decarboxylase (UROD) and coproporphyrinogen oxidase (CPO).

5. The composition of claim 1, wherein the genes include aminolevulinic acid synthase (ALAS), aminolevulinic acid dehydratase (ALAD), uroporphyrinogen decarboxylase (UROD), coproporphyrinogen oxidase (CPO), protoporphyrinogenidase (PPO), and porphobilinogen deaminase (PBGD).

6. The composition of claim 1, wherein the genes include aminolevulinic acid synthase (ALAS), aminolevulinic acid dehydratase (ALAD), uroporphyrinogen decarboxylase (UROD), coproporphyrinogen oxidase (CPO), protoporphyrinogenidase (PPO), porphobilinogen deaminase (PBGD), uroporphyrinogen III synthase (UROS) and ferrochelatase (FC).

7. The composition of claim 6, wherein the genes include aminolevulinic acid synthase (ALAS) comprising a Heme Regulatory Motif (HRM) mutant.

8. The composition of claim 6, wherein the fungus is selected from Aspergillus oryzae, Neurospora intermedia, and Neurospora crassa.

9. The composition of claim 6, wherein the fungus is Aspergillus oryzae NSAR1or Aspergillus oryzae RIB40.

10. The composition of claim 6, wherein the fungus provides an increased heme levels at least 5-fold above the non-engineered background strain, as assessed by LC-MS.

11. The composition of claim 6, wherein the fungus provides an increased heme level at least 10-fold above the non-engineered background strain, as assessed by LC-MS.

12. The composition of claim 6, wherein the fungus provides an increased heme level at least 15-fold above the non-engineered background strain, as assessed by LC-MS.

13. The composition of claim 6, wherein the fungus is engineered to express a hemoglobin protein, which acts as a sink for elevated heme inside the fungus.

14. The composition of claim 13, wherein the fungus is engineered to express a hemoglobin protein that is a Leghemoglobin from soy, alfafa or clove.

15. The composition of claim 13, wherein the fungus is engineered to express a hemoglobin protein that is a flavohemoglobin from Neurospora crassa, Aspergillus oryzae, Saccharomyces or cerevisiae.

16. The composition of claim 13, wherein the fungus is engineered to express a hemoglobin protein from cow, pig, chicken, salmon or tuna.

17. The composition of claim 6, engineered with flavor components sufficient to mimic flavors and textures of a meat selected from beef, chicken or pork.

18. The composition of claim 6, formatted as a mimic or substitute of a meat product selected from ground beef, beef steak, bacon and sausage.

19. The composition of claim 6, wherein the fungus is growable on an inexpensive substrate, such as cellulosic biomass, molasses or food waste.

20. A method of making an edible filamentous fungus, the method comprising genetically engineering the fungus to overexpress heme biosynthesis genes to elevate heme levels above a corresponding non-engineered level.
